(19) United States
(12) Patent Application Publication
(10) Pub. No.: US 2002/0164322 A1
(43) Pub. Date: Nov. 7, 2002

(54) Suspension comprising fibrinogen, thrombin and alcohol, a method for preparing such a suspension, a method for coating a carrier with such a suspension, a method of drying a coating of a carrier, and a coated collagen sponge

(76) Inventor: Alfred Schaufler, Puchenau (AT)

Correspondence Address: WENDEROTH, LIND & PONACK, L.L.P., 2033 K STREET N. W., SUITE 800, WASHINGTON, DC, 20006-1021, US

(21) Appl. No.: 10/054,889

(22) Filed: Jan. 25, 2002

Related U.S. Application Data
(60) Provisional application No. 60/263,699, filed on Jan. 25, 2001.

(30) Foreign Application Priority Data
Jan. 25, 2001 (DK) PA 2001 00135

(51) Int. Cl. A61L 2/00; B05D 3/00; A61K 38/48

(52) U.S. Cl. 424/94.63; 427/2.1; 427/372.2

(57) ABSTRACT

A suspension of fibrinogen, thrombin, alcohol and optionally aprotinin is obtained by mixing fibrinogen in alcohol with thrombin in alcohol. The suspension contains fibrinogen and thrombin particles with a Folk Ward mean diameter of 25-100 μm. The thrombin may be human, bovine or recombinant. The fibrinogen may be human or recombinant. A method for coating a carrier, such as a collagen sponge, with the suspension, and a method for drying the coating is disclosed. The coated collagen carrier may be used as a ready-to-use absorbable composition for tissue gluing, tissue sealing and haemostasis wherein the carrier is coated with solidly fixed components of fibrin glue, i.e. fibrinogen and thrombin.

### TECHNICAL FIELD

The present invention relates to a suspension comprising fibrinogen, thrombin, an alcohol and optionally aprotinin. The invention further relates to a method for preparing such a suspension and to a method for coating a carrier with such a suspension. The carrier may be a collagen carrier, such as a collagen sponge. The invention further relates to a method of drying a coated carrier, in particular a collagen carrier coated with a suspension according to the invention, and thereby obtained coated collagen carrier having the active substances solidly fixated to the carrier.

The coated collagen carrier may be used as a ready-to-use absorbable composition for tissue gluing, tissue sealing and haemostasis consisting essentially of a carrier coated with solidly fixed components of fibrin glue: fibrinogen and thrombin. This fixed combination can be applied directly to e.g. a wound surface. Upon contact with blood, body fluids or physiological saline, the mechanism of this system mimics the final stage of the coagulation cascade, in which thrombin catalyses the conversion of fibrinogen to fibrin and the activation of factor XIII to give XIIIa. Faktor XIIIa, once formed, stabilises the fibrin clot by covalent cross-linking.

Like a two-component adhesive, wound surface and carrier are glued together by polymerisation. During this process, which lasts approximately 3 to 5 minutes, the coated collagen carrier of the invention is preferably pressed onto the wound area. The components of the composition of the invention are degraded enzymatically in about 4-6 months after application.

### BACKGROUND OF THE INVENTION

Commercial fibrin glues, that mimic the last step of the coagulation cascade, consist of a highly concentrated fibrinogen solution to be mixed with a thrombin solution before application to the surgical wound exist. These mixtures contain a fibrinolysis inhibitor, e.g. aprotinin or ε-aminocaproicacid, to prevent premature dissolution of the fibrin clot by the fibrinolytic enzyme plasmin. These two-component fibrin glues are valuable in various surgical procedures but may be washed away before haemostasis is achieved if the bleeding is heavy. The two-component fibrin glues furthermore need some preparatory steps including thawing or dissolution. Thus, they are rather impractical and cumbersome to work with and experience is needed for successful use of these fibrin glues.

During the last decade numerous fibrin sealants became the methods of choice in surgery in a number of indications. However, in the majority of trials with fibrin glues a collagen fleece was additionally used to improve haemostatic and adhesive features, indicating their disadvantages and their restrained use by the surgeons.

Collagen has been used as a haemostatic agent since the late sixties. Collagen is the most frequent structural protein in all mammalians. The monomeric protein of approximately 300 kDa (tropocollagen) is covalently crosslinked at specific sites. The mature protein is therefore insoluble and forms characteristic fibrils with high tensile strength. Numerous sub-classes of collagen have been described, the most common of which is collagen type I, the main collagen type in skin, tendons bones and cornea. Collagen is a fibrous protein consisting of a triple helix with a length of approximately 290 nm. Five of these triple helices (tropocollagen molecules) are staggered to form a microfibril with a diameter of approximately 3.6 nm. These microfibrils have polar and non-polar segments that are readily accessible for specific inter- and intrafibrillar interactions. Microfibrils are packed into a tetragonal lattice to form subfibrils with a diameter of about 30 nm. These subfibrils are then assembled into the collagen fibril, the basic unit of connective tissue, which has a diameter of several hundred nm and is therefore visible in the light microscope as a thin line.

Collagen may be used as a material for sealing wounds, possibly with a coating comprising a fibrin glue. Fibrin glues, i.e. the combination of fibrinogen, thrombin and aprotinin, have successfully been used therapeutically for many years for gluing tissues and nerves and for sealing surfaces when there is minor bleeding. One drawback of the fibrin glues has been that in case of major bleeding the glue is usually washed away before sufficient polymerisation of fibrin has occurred. To overcome this problem surgeons have begun applying manually liquid fibrin glues to absorbable carriers such as collagen fleece.

Despite the impressive success of these combined applications this method has not been applied on a broad scale, due to some disadvantages. The preparation is relatively cumbersome, the method requires experience and skilled personnel, and the preparation is not readily available in cases of emergency, the time for preparation being in the range of 10 to 15 min. These factors stimulated the development of an improved product resulting in the development of a fixed combination of a collagen carrier covered with a coating of solid fibrinogen, solid thrombin and solid aprotinin as disclosed in EP 0 059 265.

The function of the collagen carrier disclosed in EP 0 059 265 is mainly that of a carrier which adsorbs and confers mechanical stability to the coagulation preparation with which it is coated.

A product that combines the haemostatic features of fibrin glue with the asset of collagen as a carrier has been developed and manufactured under the trademark TachoCombe®. TachoComb® is a ready-to-use and easily applicable fixed combination of a collagen patch coated with the following active components of fibrin glue: human fibrinogen, bovine thrombin and bovine aprotinin. TachoComb® has been sold since the early 1990s by Nycomed Pharma and has been used in clinical trials in Europe in more than 2500 patients. The product has furthermore been used in more than 700 patients in the Japanese clinical programme in a large variety of indications such as liver and lung resections, surgery of the biliary tract, splenic, renal and pancreatic surgery, ENT surgery, gynaecological surgery, and vascular surgery. TachoCombe® was found to be effective and safe.

No clinical complications related to the application of TachoComb® have been reported in the course of the clinical trials performed.

In WO97/37694 (Immuno France S. A.) it is disclosed in reference example 4 that when a collagen product or TachoComb® was used, there was no haemostasis leading to bleeding to death when TachoComb® was used in contrast to haemostasis within 5 minutes when a collagen product without a thrombin content prepared according to WO97/37694 was prepared.

In WO96/40033 the disadvantages of the bovine thrombin used in TachoComb® are emphasized in that the use of bovine or other species of thrombin can introduce harmful viral contamination and possible transmission of bovine diseases, such as bovine spongiform encephalitis.

U.S. Pat. No. 6,177,126 B1 discloses a device and a process for the production of a material for sealing and healing wounds. The device comprises a container having, at its bottom part, two perforated plates which are movable relative to each other, so as to allow a suspension contained in the container to drip onto a carrier which is moving past the container under the bottom part thereof.

### DESCRIPTION OF THE INVENTION

It is an object of the invention to provide an improved suspension which is suitable, e.g., for use as a coating for a collagen carrier, with the aim of providing a ready-to-use absorbable composition for tissue gluing, tissue sealing and haemostasis. It is a further object of the invention to provide a method for producing such a suspension. It is a still further object of the invention to provide an improved method of coating a carrier, such as a collagen carrier, with a suspension containing fibrinogen and thrombin. A further object of the invention is to provide a method of drying a wet coating of the suspension applied to a carrier, with the aim of ensuring a satisfactory fixation of the coating to the carrier. It is a still further object of the invention to provide a coated collagen sponge with a coating of fibrinogen and thrombin which efficiently mimics the final stage of the coagulation cascade, once the coated collagen sponge has been brought into contact with blood, body fluids or physiological saline. Further, it is an object of the invention to provide a coated collagen sponge with the above coating which has a sufficient fixation of the coating to the collagen sponge, i.e. a satisfactory low abrasion of the coating when submitted to mechanical impact.

In a first aspect the invention provides a suspension comprising fibrinogen, thrombin and alcohol, the suspension having been obtained by a method comprising:

providing a fibrinogen mixture of fibrinogen and an alcohol,

providing a thrombin mixture of thrombin and an alcohol,

mixing the fibrinogen mixture and the thrombin mixture, so as to obtain said suspension, the suspension containing fibrinogen and thrombin particles, the Folk Ward mean diameter of the particles being 25-100 μm, such as 35-80 μm, such as 40-78 μm, such as 40-75 μm, such as 45-60 μm, such as 47-55 μm, or such as 60-100 μm, such as 60-80 μm, such as 65-75 μm, preferably within +/−5 μm, such as within +/−4 μm, such as within +/−3.5 μm, such as within +/−2 μm, such as within +/−1.5 μm, such as within +/−1 μm, such as within +/−0.8 μm, such as within +/−0.6 μm, such as within +/−0.5 μm. It has been found that such a suspension when coated onto a carrier, such as a collagen carrier, is efficient in a ready-to-use absorbable composition for tissue gluing, tissue sealing and haemostasis. The suspension may optionally comprise aprotinin, added to the fibribinogen mixture as a concentrated aqueous solution. Riboflavin may be added as a colorant, so that the suspension may easily be identified once it has been coated onto a carrier and dried.

Due to the physical property of the suspension, especially the sedimentation behaviour of the rather large particles in an alcohol, no standard liquid viscosity measure of the suspension is possible. Thus, an alternative method for providing viscosity measure has been implemented. Accordingly, the suspension may have a viscosity so that a volume of 90-120 ml of suspension, when influenced by gravity only, exits through a bottom opening of a container having:

a cylindrical portion with an inner diameter of 40-50 mm and an inner height of 55-65 mm, and

a conical bottom portion with a height of 17-23 mm, whereby the bottom opening is provided at the lower end of the conical portion as a circular opening with a diameter of 2-3 mm,

in 25-75 seconds.

In case of the container being made from steel, the container and the opening having the following dimensions:

inner diameter of the cylindrical portion: 46 mm,

an inner height of the cylindrical portion: 60 mm,

inner height of the conical bottom: 20.5 mm,

inner diameter of the bottom opening: 2.6 mm,

length of passage connected to the bottom opening: 9 mm, or

in case of the container being made from a plastic material, the container and the opening having the following dimensions:

inner diameter of the cylindrical portion: 50 mm,

an inner height of the cylindrical portion: 41 mm,

inner height of the conical bottom: 24 mm,

inner diameter of the bottom opening: 2.5 mm,

length of passage connected to the bottom opening: less than 5 mm,

the above exit time for the suspension may be 25-60 seconds, such as 25-50 seconds, such as 30-50 seconds, such as 32-44 seconds, such as 34-38 seconds.

The parameters and features of the suspension disclosed below in connection with the method of the second aspect of the invention also apply to the suspension of the first aspect of the invention.

In a second aspect the invention provides a method of preparing a suspension with fibrinogen and thrombin, comprising:

providing a fibrinogen mixture of fibrinogen and an alcohol,

providing a thrombin mixture of thrombin and an alcohol,

mixing the fibrinogen mixture and the thrombin mixture, so as to obtain said suspension,

so as to obtain a suspension containing fibrinogen and thrombin particles, the Folk Ward mean diameter of the particles being 25-100 μm. The parameters and features disclosed above in connection with the suspension according to the first aspect of the invention also apply to the method of the second aspect of the invention.

At the step of providing the fibrinogen mixture, the fibrinogen may be pre-micronised by a suitable method, e.g. sieving, to obtain particles having a Folk Ward mean diameter of 25-100 μm. The micronised fibrinogen may, for example, be stirred into the alcohol to obtain said fibrinogen mixture. At the step of providing the mixture, the fibrinogen may be also directly homogenized in an alcohol, preferably at a temperature between 0° C. and 12° C., such as between 2° C. and 8° C. The temperature may be lowered during homogenization. The step of mixing the fibrinogen mixture and the thrombin mixture may be carried out while stirring the suspension, whereby the stirring may be carried out at a temperature between 0° C. and 12° C., such as between 2° C. and 8° C.

The thrombin may comprise human thrombin, bovine thrombin, or recombinant thrombin, and the fibrinogen may comprise human fibrinogen or recombinant fibrinogen. The alcohol may be an organic alcohol, such as methanol, ethanol, propanol, isopropanol, such as an anhydrous organic alcohol, an anhydrous ethanol, an anhydrous propanol or an anhydrous isopropanol. Human fibrinogen may be supplied in a solid freeze-dried form.

In a third aspect the invention provides a method for coating a carrier with a suspension comprising fibrinogen and thrombin, wherein the suspension has been derived from a method comprising the steps of:

providing a fibrinogen mixture of fibrinogen and an alcohol,

providing a thrombin mixture of thrombin and an alcohol,

mixing the fibrinogen mixture and the thrombin mixture, so as to obtain said suspension,

so as to obtain a suspension containing fibrinogen and thrombin particles, the Folk Ward means diameter of the particles being 25-100 μm, the method of coating comprising:

providing the suspension of fibrinogen, thrombin and an alcohol at a location near the carrier,

applying said suspension to a coating surface of the carrier.

The carrier may be a collagen carrier, such as a collagen sponge. The collagen sponge may fulfil at least one and preferably a plurality of the following criteria:

pH-value between 5.0 and 6.0,

lactic acid content at the most 5%,

ammonium content at the most 0.5%,

soluble protein content, calculated as albumin content, at the most 0.5%,

sulphate ashes content at the most 1.0%,

heavy metal content at the most 20 ppm,

microbiological purity, at the most 10³ CFU/g,

collagen content of 75 to 100%,

density of 1 to 10 mg/cm³,

elasticity module in the range of 5-100 N/cm.

The collagen sponge may be derived from a method comprising the steps of:

preparing a collagen gel,

mixing air into the collagen gel, so as to obtain a collagen foam,

drying the collagen foam, so as to obtain a dry block of carrier having chambers therein,

isolating, from the block of collagen sponge, parts of sponge with a chamber diameter of more than 0.75 mm and less than 4 mm, or with chambers of an average diagonal dimension of 3 mm.

In the present context, the term “chamber diameter” should be understood as the largest straight-line walt-to-wall distance in a chamber, i.e. the largest diagonal straight-line distance of a chamber. The chambers may be of a polygonal shape, such as of an octagonal shape. It has been found that a chamber diameter of more than 0.75 mm and less than 4 mm, or a chamber diameter of at most 3 mm, renders the collagen sponge particularly useful for being coated with a suspension containing fibrinogen and thrombin. It has further been found that a coated collagen sponge prepared by the above method is air and liquid tight in the sense that, once the coated collagen sponge has been applied to a wound, it will not allow air or liquid to soak through the collagen sponge.

The step of applying the suspension to the carrier may be performed at an ambient temperature of 0°-12° C., such as at 1°-10° C., such as at 2°-8° C. Further, the step of applying the suspension to the carrier may be carried out in an ambient atmosphere with a relative humidity of 75-99%, such as 85-95%. A volume of 0.08 ml-0.12 ml of suspension is preferably applied to the carrier pr. cm² of the coating surface. To ensure a homogeneous efficacy of the final coated carrier across its whole surface, the suspension is preferably distributed evenly over a given width of the coating surface, so that the mass of fibrinogen per area unit of the coating surface varies at most 25%, such as at most 20%, such as at most 15%, such as at most 10%.

An applicator comprising at least one jet may be used for applying the suspension to the carrier, whereby the suspension is forced through the jet while the carrier and the jet are moved relative to each other. The applicator may comprise or be arranged near a conveyor belt, a stirring unit connected to a pump or a system of pumps or another supplying equipment, and a jet or a system of jets which moves transversely, e.g. at right angles to the conveyor belt. Depending on the specific characteristics of the media, the jet or the system of jets may have various shapes and sizes. The jet or the system of jets may be connected to the supplying equipment via tubes. The supplying equipment may promote the coating medium from the stirring unit to the jet systems. During the coating process the jet system may move across the carrier. In its waiting position it may hold on one side of the conveyor belt. The coating process may be initiated by a light barrier sensing the presence of a carrier on the conveyor belt, and may likewise be stopped by a light barrier signal. Such an applicator confers a relatively small dead volume, and it is easy to handle, including easy to clean. Furthermore, it confers the possibility to interrupt the coating process at any time, it is applicable in a relatively broad range of viscosities, and it confers a homogenous coating.

Alternatively, or additionally, an applicator comprising a container having a plurality of separate outlets may be used for applying the suspension to the carrier, whereby the suspension is forced from the container through the outlets onto the carrier. The latter of type of applicator in the form of a container having movable plates at its bottom is disclosed in U.S. Pat. No. 6,177,126 B1 which is hereby incorporated by reference in its entirety. Due to the even distribution conferred by the devices of U.S. Pat. No. 6,177,126 B1, one of those devices are applied in a preferred embodiment of the invention. The carrier and the applicator are preferably moved relative to each other in a transport direction while the suspension is being applied to the carrier, whereby the rate of movement may be 0.025 m/s-0.05 m/s, such as 0.03-0.04 m/s. The flow rate of suspension applied to the carrier through the applicator may be 400-600 ml/min, such as 470-550 ml/min, such as 495-505 ml/min.

In a fourth aspect the invention relates to a method of drying a suspension of fibrinogen, thrombin and an alcohol applied as a wet coating on a coating surface of a carrier, the method comprising the step of submitting the coated carrier to a pressure below 1000 mbar, so as to obtain a dried coating surface on the carrier, so as to fixate the dried coating to the coating surface. By applying a vacuum and using the vacuum in the drying process, a low temperature (2-10° C.) and a high relative humidity (80-95%) may be kept, whereby the structure and the physical properties of the carrier, in particular a carrier in the form of a collagen, such as a collagen sponge, as well as of the fibrinogen and thrombin may be maintained.

The suspension may be obtained by:

providing a fibrinogen mixture of fibrinogen and an alcohol,

providing a thrombin mixture of thrombin and an alcohol,

mixing the fibrinogen mixture and the thrombin mixture, so as to obtain said suspension,

and the carrier may be a collagen sponge which has been derived from a method comprising the steps of:

preparing a collagen gel,

mixing air into the collagen gel, so as to obtain a collagen foam,

drying the collagen foam, so as to obtain a dry block of collagen sponge having chambers therein,

isolating, from the block of collagen sponge, parts of sponge with a chamber diameter of more than 0.75 mm and less than 4 mm, or with a chamber with an average diagonal dimension of 3 mm,

and the coating may be applied to the collagen sponge by:

providing the suspension of fibrinogen, thrombin and an alcohol at a location near the collagen sponge,

applying the suspension to the coating surface of the collagen sponge.

The methods for preparing the collagen sponge and for preparing the suspension are discussed above in connection with the methods of the second and third aspects of the invention.

During drying the coated carrier may be submitted to said pressure at a temperature of 0° C.-12° C., such as 1° C.-10° C., such as 2° C.-8° C., and/or at a relative humidity of the surrounding atmosphere of 75-99%, such as 85-95%. A flow of air may pass across the coated carrier during drying, so as to convey vapor away from the coated carrier.

In order for the drying to complete, the coated carrier is preferably kept at said conditions for a period of at least 1 hour, such as at least 2 hours, such as at least 4 hours.

Due to shrinkage, the area of the dried coating surface is smaller than the size of the area of the wet coating surface. In the method according to the invention, the area of the dried coating surface is at least 75% the size of the area of the wet coating surface, such as least 80%.

In order to keep the active components stable when the coated carrier is stored, the carrier and the dried coating surface together preferably have a water content not exceeding 12% by weight, such as not exceeding 8% by weight. Any parameters and features of the suspension and the collagen sponge, including there methods of manufacture, discussed in connection with the other aspects of the invention also apply to the method of the fourth aspect of the invention.

In a fifth aspect the invention relates to a coated collagen sponge with a coating of fibrinogen and thrombin, wherein the coated collagen sponge has been obtained by a method comprising the steps of:

providing a collagen sponge by a method comprising:

preparing a collagen gel,

mixing air into the collagen gel, so as to obtain a collagen foam,

drying the collagen foam, so as to obtain a dry block of collagen sponge having chambers therein,

isolating, from the block of collagen sponge, parts of sponge with a chamber diameter of more than 0.75 mm and less than 4 mm, or with chambers of an average diagonal dimension of 3 mm,

applying a suspension of fibrinogen, thrombin and an alcohol to a coating surface of the collagen sponge, and

submitting the coated carrier to a pressure below 1000 mbar, so as to obtain a dried coating surface on the carrier, so as to fixate the dried coating to the coating surface, the coated collagen sponge having at least one of the following properties:

the suspension is distributed evenly over a given width of the coating surface, so that the mass of fibrinogen per area unit of the coating surface varies at most 25%,

the abrasion of the coating is less than 2.0 mg/cm² when a sample of the coated material is shaken on a Vibrofix shaker at a frequency of 800-1200 rpm for 2 minutes.

The even distribution of the suspension over the coating surface improves the efficacy of the coated surface when applied, e.g. for tissue gluing, tissue sealing or haemostasis. The low abrasion of the coating ensures that the coated collagen sponge may be transported, grabbed by a surgeon's hands and/or by a surgical instrument and otherwise handled without loosing the dried suspension, i.e. the coating. The fibrinogen formulation may account for approximately 60-90% of the total weight of the coated collagen sponge. The formulation usually contains about 50-600% of weight of the following substances: salts, amino acids and albumin. Fibrinogen alone usually constitutes 40-50% of the formulation.

The suspension preferably has a water content of 20-80 mg/ml, such as 24-32 mg/ml. The thrombin content of the suspension may be 20-40 I.U./ml, such as 24-33 I.U./ml. In average, the thrombin content after coating may be 2-4 I.U./cm² over the coating surface, such as 2.3-3.3 I.U./cm². It may be desirable that the thrombin content does not exceed 5 I.U./cm² at any location on the coating surface, or that it does not exceed 3.8 I.U./cm² at any location on the coating surface.

The microbiological purity of the coated carrier preferably is at most 4 CFU/cm², such as at most 2.25 CFU/cm².

In a further independent aspect the invention relates to the use of the above-mentioned coated collagen sponge for tissue gluing, tissue sealing and haemostasis.

### BRIEF DESCRIPTION OF THE DRAWINGS

FIGS. 1-7 disclose various coated carriers and instruments for applying them, as discussed in Example VIII.

FIG. 8 is a flow chart illustrating a chain of sub-processes from producing a suspension to packing a coated collagen sponge,

FIG. 9 is an illustration of devices used in obtaining a measure for the viscosity of the suspension,

FIGS. 10 and 11 contain a flow chart illustrating a process for obtaining a collagen sponge.

### DETAILED DESCRIPTION OF THE DRAWINGS

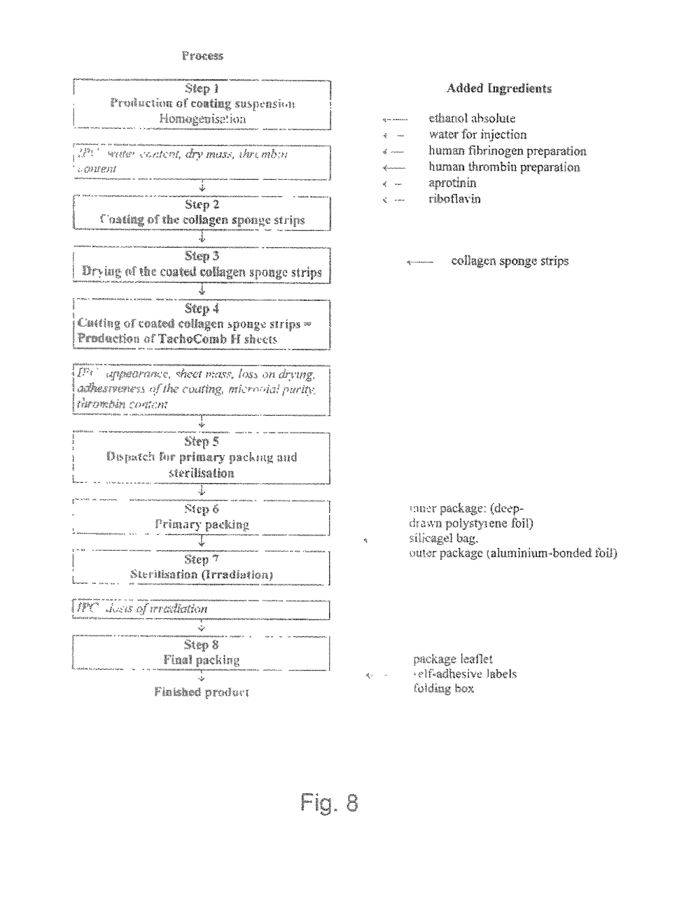

Preferred embodiments of the methods and products of the present invention are described below, cf. also FIG. 8.

A suspension comprising fibrinogen, thrombin and alcohol may be produced by the method for producing a suspension according to the invention, as follows:

Fibrinogen is homogenised in a 100% ethanol at 2-8° C., resulting in a mixture of fibrinogen and alcohol, the mixture constituting approximately 80% of the volume of the final suspension volume. Then, riboflavin is added. The mixture is subsequently stirred in a closed vessel until further processing thereof.

Human or bovine thrombin is dissoluted with water for injection. The solution is added to a 35 fold amount of 100% ethanol at 0-8° C. The thereby achieved thrombin suspension is homogenised at 0-8° C. for 80-100 sec.

Before the fibrinogen and thrombin mixtures are mixed, an aprotinin solution and water for injection are added to the fibrinogen mixture. Then, the thrombin mixture is added to the fibrinogen mixture. Final volume of suspension is prepared by adding a 100% ethanol at 2-8° C.

Methods comprising the above steps are hereinafter referred to as “group I methods”.

As an alternative, the method for producing a suspension according to the invention, comprising fibrinogen, thrombin and alcohol, comprises the following steps:

The fibrinogen mixture is obtained by adding pre-micronised fibrinogen of a particle size of 35-80 μm Folk Ward mean diameter and riboflavin while stirring to a 94-97% ethanol at 2-8° C. The thereby resulting mixture of riboflavin and ethanol constitutes approximately 70-80% of the final suspension volume. The fibrinogen mixture is further stirred in a closed vessel until further processing thereof.

The thrombin mixture is obtained by adding thrombin to a 94-97% ethanol at −30° C. The thereby achieved thrombin mixture is homogenised for 80-100 sec. Alternatively, the thrombin mixture is obtained by solving thrombin in water for injection, and subsequently the thereby obtained thrombin solution is slowly added to 17-35 fold amount of 100% ethanol at −30° C. The suspension is homogenised 80-100 sec.

An UltraTurrax equipment by IKA may be used as a homogenizing equipment.

The thrombin mixture is added to the mixture containing fibrinogen and riboflavin. An 94-97% ethanol at 2-8° C. is added. Methods comprising the above steps are hereinafter referred to as “group II methods”.

The above group I and group II methods may result in a suspension according to the invention, preferably with the following characteristics:

Ethanol concentration: 94-97%

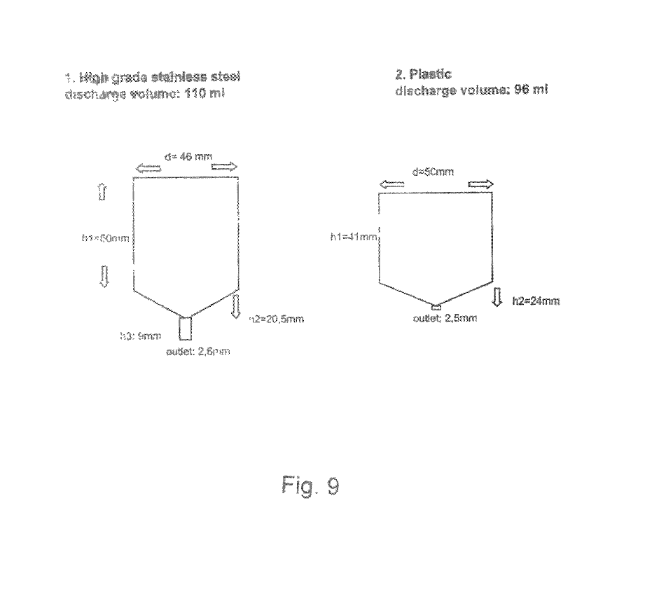

Exiting time measured with the apparatus depicted in to the left in FIG. 9: 31.5-48 seconds

Sedimentation behaviour: Volume of solid particles in percentage of total volume:

5 minutes after start of test: more than 85%

24 hours after start of test: 50-80%

Particle size: 35-80 μm Folk Ward mean diameter.

In one embodiment of the method of drying according to the invention, collagen sponge strips are incubated at 2-8° C. at 80-91% relative humidity for 2-30 hours, before coating of a carrier in the form of a collagen sponge. The applicator for applying the suspension to the collagen sponge is described above. Once coated, the collagen sponge strips are incubated at 2-8° C. and 80-90% relative humidity for 8-60 minutes. The coated collagen sponge strips are dried in a vacuum drying chamber at an air temperature of 2-80° C., 80-90% relative humidity. An air flow is passed over the collagen strips through an aspiration valve, at an air flow rate of 1,2-40 m³ per hour. A vacuum of 30-60 mbar is applied, i.e. an absolute pressure of approximately 970 mbar, depending upon atmospheric pressure, and the coated collagen strips are dried for 2-5 hours.

A measure of the viscosity of the suspension is obtained by use of one of the devices depicted in FIG. 9 and as described above. The device shown to the left in FIG. 9 is made from steel, and the container and the bottom opening have the following dimensions:

inner diameter of the cylindrical portion: 46 mm,

an inner height of the cylindrical portion: 60 mm,

inner height of the conical bottom: 20.5 mm,

inner diameter of the bottom opening: 2.6 mm,

length of passage connected to the bottom opening: 9 mm.

The device shown to the right in FIG. 9 is made from a plastic material, and the container and the bottom opening have the following dimensions:

inner diameter of the cylindrical portion: 50 mm,

an inner height of the cylindrical portion: 41 mm,

inner height of the conical bottom: 24 mm,

inner diameter of the bottom opening: 2.5 mm,

length of passage connected to the bottom opening: less than 5 mm.

Fibrinogen Raw-materials

| | % of total substance | | |
|---|---|---|---|
| Component | Formulation A | Formulation B | Formulation C |
| Human fibrinogen | 36-52 | 42-47 | 36-52 |
| Human albumin | 16-24 | 20-24 | 16-24 |
| Total protein | 52-76 | 62-71 | 52-76 |
| Sodium chloride | 8-14 | 0 | 8-14 |
| tri Sodium citrate | 2-4 | 1-3 | 2-4 |
| Arginine | 15-26 | 15-21 | 15-26 |
| (hydrochloride) | | | |
| Glycine | 0 | 6-9 | 1-2 |
| Histidine | 0 | 3-5 | 0 |
| Sucrose | 0 | 0 | 1-2 |
| Residual moisture | <=2 | 2-4 | <=1, 5 |

Thrombin Raw-materials

| | Activity | | |
|---|---|---|---|
| | (I.U/mg) | Residual | additional |
| Formulation | substance | moisture | substances additives |
| Human thrombin A | 360-540 | <=3% | Human albumin, sodium |
| | | | chloride, sodium citrate |
| Bovine thrombin A | >400 | <=3% | Bovine albumin, sodium |
| | | | chloride, sodium citrate |
| Human thrombin B | 7-10 | <=3% | Human albumin, sodium |
| | | | chloride, sodium citrate |
| Human thrombin C | 35-60 | <=3% | Human albumin, sodium |
| | | | chloride, sodium acetate, |
| | | | glycine |

Examples I-VI below illustrate various procedures for preparation of a coated collagen sponge with a coating of fibrinogen and thrombin according to the invention. The procedures include methods of preparing a suspension according to the invention, resulting, in the embodiments described below, in suspensions according to the invention. Further, methods for coating according to the invention and methods for drying according to the invention are applied.

### EXAMPLE I

In the present example, the suspension contains human fibrinogen formulation B and human thrombin formulation B.

A final suspension volume of 3500 ml was obtained by a group II method by applying the following quantities and parameters:

Fibrinogen Mixture:

2800 ml ethanol (94% at 2° C.-8° C.)

492.5 g micronised human fibrinogen formulation B

493.5 mg riboflavin

The fibrinogen mixture was stored for 20 hours at 2-8° C. while being stirred.

Thrombin mixture:

100 ml ethanol (100% at −30° C.)

12,27 g human thrombin formulation B

The thrombin mixture was stored for 18hours at −30° C.

Suspension:

157 ml of thrombin mixture are added to the fibrinogen mixture.

A 94% ethanol at 2-8° C. was added to fill to the final suspension volume of 3500 ml.

Suspension Characteristics:

1. Ethanol concentration: 94.3%

2. Exiting time measured with the steel apparatus depicted to the left in FIG. 9: 36.5 seconds

3. Sedimentation behaviour:

a) sedimentation volume 5 minutes after start: 98% of test volume,

b) sedimentation volume 24 hours after start: 64% of test volume.

4. Particle size (Folk Ward mean diameter): 56.4+/−1.3 μm

Carriers in the form of collagen strips were coated with the suspension. First, 48 collagen sponge strips were pre-incubated in a cooling chamber, at the following conditions:

Temperature: 5.2° C.

Absolute humidity: 4.8 g water per kg air

Incubation time: 18.5 hours

An applicator as disclosed in U.S. Pat. No. 6,177,126 B1 was used for coating the collagen sponge strips with the suspension.

The coated collagen sponge strips were dried as follows:

The coated strips were incubated for 15 minutes at a temperature of 5.2° C. and an absolute humidity of 4.8 g water per kg air.

The coated strips were then dried in a vacuum drying chamber at the following drying conditions:

Air condition: temperature of 5.2° C., absolute humidity of 4.8 g water per kg air

Airflow through aspiration valve: 23 m³ per hour

Vacuum: 59 mbar

Drying time: 4 hours

The abrasion of the obtained coating on the collagen sponge strips was approximately 0.2 mg/cm² when a sample of 1×5 cm² is shaken in a test-tube on a Vibrofix shaker at a frequency of 800-1200 rpm for 2 minutes.

### EXAMPLE II

In the present example, the suspension contains human fibrinogen formulation C and human thrombin formulation C.

A final suspension volume of 3500 ml was obtained by a group II method by applying the following quantities and parameters:

Fibrinogen Mixture:

2252 ml ethanol (94% at 2° C.-8° C.)

370.7 g micronised human fibrinogen formulation C

493.5 mg riboflavin

The fibrinogen mixture was stored for 20 hours at 2-8° C. while being stirred.

Thrombin Mixture:

188 ml ethanol (1000% at −30° C.)

12 vials human thrombin formulation C /12 ml water for injection

The thrombin mixture was stored for 18 hours at −30C.

Suspension:

164.5 ml of thrombin mixture were added to the fibrinogen mixture.

A 94% ethanol at 2-80° C. was added to fill to the final suspension volume of 3500 ml.

Suspension Characteristics:

1. Ethanol concentration: 94.1%

2. Exiting time measured with the steel apparatus depicted in FIG.: 32.8 seconds

3. Sedimentation behaviour:

a) sedimentation volume 5 minutes after start: 94% of test volume,

b) sedimentation volume 24 hours after start: 71% of test volume.

4. Particle size (Folk Ward mean diameter): 49.2+/−0.93 μm

Carriers in the form of collagen strips were coated with the suspension. First, 48 collagen sponge strips were pre-incubated in a cooling chamber, at the following conditions:

Temperature: 4.8° C.

Relative humidity: 90.30%

Incubation time: 22.25 hours

An applicator as disclosed in U.S. Pat. No. 6,177,126 B/1 was used for coating the collagen sponge strips with the suspension.

The coated collagen sponge strips were dried as follows:

The coated strips were incubated for 13 minutes at a temperature of 4.9° C. and an absolute humidity of 4.8 g water per kg air.

The coated strips were then dried in a vacuum drying chamber at the following drying conditions:

Air condition: temperature of 5.2° C., absolute humidity of 4.9 g water per kg air

Air flow through aspiration valve: 25 m³ per hour

Vacuum: 60 mbar

Drying time: 4 hours

The abrasion of the obtained coating on the collagen sponge strips was approximately 0.2 mg/cm² when a sample of 1×5 cm² is shaken in a test-tube on a Vibrofix shaker at a frequency of 800-1200 rpm for 2 minutes.

### EXAMPLE III

In the present example, the suspension contains human fibrinogen formulation B and human thrombin formulation B, and aprotinin.

A final suspension volume of 1000 ml was obtained by a group II method by applying the following quantities and parameters:

Fibrinogen Mixture:

820 ml ethanol (100% at 2° C.-8° C.), 39.4 ml water for injection, and 10.6 ml aprotinin

90.67 g micronised human fibrinogen formulation B

141 mg riboflavin

The fibrinogen mixture was stored for 20 hours at 2-8° C. while being stirred.

Thrombin Mixture:

50 ml ethanol (100% at −30° C.)

3.75 g human thrombin formulation B

The thrombin mixture was stored for 16hours at −30° C.

Suspension:

The total volume of thrombin mixture was added to the fibrinogen mixture.

A 100% ethanol at 2-8 C. was added to fill to the final suspension volume of 1000 ml.

Suspension Characteristics:

1. Ethanol concentration: 95%

2. Exiting time measured with the plastic apparatus depicted in to the right in FIG. 9: 35 seconds

3. Sedimentation behaviour:

a) sedimentation volume 5 minutes after start: 89% of test volume,

b) sedimentation volume 24 hours after start: 76% of test volume.

4. Particle size (Folk Ward mean diameter): 74.4+/−3.5 μm

Carriers in the form of collagen strips were coated with the suspension. First, 16 collagen sponge strips were pre-incubated in a cooling chamber, at the following conditions:

Temperature: 5.0° C.

Relative humidity: 85%

Incubation time: 17 hours

An applicator as disclosed in U.S. Pat. No. 6,177,126 B1 was used for coating the collagen sponge strips with the suspension.

The coated collagen sponge strips were dried as follows:

The coated strips were incubated for 35 minutes at a temperature of 5° C. and a relative humidity of 85%.

The coated strips were then dried in a vacuum drying chamber at the following drying conditions:

Air condition: temperature of 5° C., relative humidity 85%

Air flow through aspiration valve: 1.2 m³ per hour

Vacuum: 35 mbar

Drying time: 4 hours

The abrasion of the obtained coating on the collagen sponge strips was approximately 0.2 mg/cm² when a sample of 1×5cm² is shaken in a test-tube on a Vibrofix shaker at a frequency of 800-1200 rpm for 2 minutes.

### EXAMPLE IV

In the present example, the suspension contains human fibrinogen formulation C and human thrombin formulation C.

A final suspension volume of 780 ml was obtained by a group II method by applying the following quantities and parameters:

Fibrinogen Mixture:

700 ml ethanol (94% at 2° C.-8° C.)

84.42 g micronised human fibrinogen formulation C

110 mg riboflavin

The fibrinogen mixture was stored for 20 hours at 2-8° C. while being stirred.

Thrombin mixture:

35 ml ethanol (100% at −30° C.)

0.54 g human thrombin formulation C

The thrombin mixture was stored for 16 hours at −30° C.

Suspension:

23.0 ml of thrombin mixture was added to the fibrinogen mixture.

A 100% ethanol at 2-8° C. was added to fill to the final suspension volume of 780 ml.

Suspension Characteristics:

1. Ethanol concentration: 94%

2. Exiting time measured with the plastic apparatus depicted to the right in FIG. 9: 33.5 seconds

3. Sedimentation behaviour:

a) sedimentation volume 5 minutes after start: 92% of test volume,

b) sedimentation volume 24 hours after start: 72% of test volume.

4. Particle size (Folk Ward mean diameter): 60.5 +/−0.5 μm

Carriers in the form of collagen strips were coated with the suspension. First, 8 collagen sponge strips were pre-incubated in a cooling chamber, at the following conditions:

Temperature: 6.0° C.

Relative humidity: 85%

Incubation time: 18.5 hours

An applicator as disclosed in U.S. Pat. No. 6,177,126 B1 was used for coating the collagen sponge strips with the suspension.

The coated collagen sponge strips were dried as follows:

The coated strips were incubated for 45 minutes at a temperature of 5° C. and a relative humidity of 85%.

The coated strips were then dried in a vacuum drying chamber at the following drying conditions:

Air condition: temperature of 5° C., relative humidity 85%

Air flow through aspiration valve: 1.2 m³ per hour

Vacuum: 35 mbar

Drying time: 4 hours

The abrasion of the obtained coating on the collagen sponge strips was approximately 0.2 mg/cm² when a sample of 1×5 cm² is shaken in a test-tube on a Vibrofix shaker at a frequency of 800-1200 rpm for 2 minutes.

### EXAMPLE V

In the present example, the suspension contains human fibrinogen formulation A and human thrombin formulation A.

A final suspension volume of 3120 ml was obtained by a group I method by applying the following quantities and parameters:

Fibrinogen Mixture:

2540 ml ethanol (100% at 2° C.-8° C.)

311.6 g human fibrinogen formulation A

440 mg riboflavin

The fibrinogen mixture was stored for 18 hours at 2-8° C. while being stirred.

Thrombin Mixture:

210 ml ethanol (100% at −30° C.)

229 human thrombin formulation A

Suspension:

87.3 ml water for injection were added to the fibrinogen mixture.

The thrombin mixture was added to the fibrinogen mixture.

A 100% ethanol at 2-8° C. was added to fill to the final suspension volume.

Suspension Characteristics:

1. Ethanol concentration: 97%

2. Exiting time measured with the steel apparatus depicted to the left in FIG. 9: 40.8 seconds

3. Sedimentation behaviour:

a) sedimentation volume 5 minutes after start: 95.6% of test volume,

b) sedimentation volume 24 hours after start: 63.5% of test volume.

4. Particle size (Folk Ward mean diameter): 51.8+/−0.8 μm

Carriers in the form of collagen strips were coated with the suspension. First, 48 collagen sponge strips were pre-incubated in a cooling chamber, at the following conditions:

Temperature: 6.5° C.

Relative humidity: 90%

Incubation time: 22.5 hours

An applicator as disclosed in U.S. Pat. No. 6,177,126 B1 was used for coating the collagen sponge strips with the suspension.

The coated collagen sponge strips were dried as follows:

The coated strips were incubated for 10 minutes at a temperature of 6.5° C. and a relative humidity of 90%.

The coated strips were then dried in a vacuum drying chamber at the following drying conditions:

Air condition: temperature of 6.5° C., relative humidity 90%

Air flow through aspiration valve: 21 m³ per hour

Vacuum: 58 mbar

Drying time: 4 hours

The abrasion of the obtained coating on the collagen sponge strips was approximately 0.2 mg/cm² when a sample of 1×5 cm² is shaken in a test-tube on a Vibrofix shaker at a frequency of 800-1200 rpm for 2 minutes.

### EXAMPLE VI

In the present example, the suspension contains human fibrinogen formulation A and bovine thrombin formulation, and aprotinin.

A final suspension volume of 16720 ml was obtained by a group I method by applying the following quantities and parameters:

Fibrinogen Mixture:

13600 ml ethanol (100% at 2° C.-8° C.)

1750.5 g human fibrinogen formulation A

2361 mg riboflavin

The fibrinogen mixture was stored for 21 hours at 2-8° C. while being stirred.

Thrombin Mixture:

420 ml ethanol (100% at -30° C.)

1229 g bovine thrombin formulation

Suspension:

162.3 ml aprotinin solution was added to the fibrinogen mixture.

304.7 ml water for injection was added to the fibrinogen mixture.

The thrombin mixture was added to the fibrinogen mixture.

A 100% ethanol at 2-8° C. was added to fill to the final suspension volume.

Suspension Characteristics:

1. Ethanol concentration: 97%

2. Exiting time measured with the steel apparatus depicted to the left in FIG. 9: 36.8 seconds

3. Particle size (Folk Ward mean diameter): 58.6+/−0.6 pm

Carriers in the form of collagen strips were coated with the suspension. First, 288 collagen sponge strips were pre-incubated in a cooling chamber, at the following conditions:

Temperature: 6.5° C.

Relative humidity: 89%

Incubation time: 25 hours

An applicator as disclosed in U.S. Pat. No. 6,177,126 B1 was used for coating the collagen sponge strips with the suspension.

The coated collagen sponge strips were dried as follows:

The coated strips were incubated for 10 minutes at a temperature of 6.5° C. and a relative humidity of 89%.

The coated strips were then dried in a vacuum drying chamber at the following drying conditions:

Air condition: temperature of 6.50° C., relative humidity 89%

Air flow through aspiration valve: 22.5 m³ per hour

Vacuum: 59 mbar

Drying time: 4 hours

The abrasion of the obtained coating on the collagen sponge strips was approximately 0.2 mg/cm² when a sample of 1×5 cm² is shaken in a test-tube on a Vibrofix shaker at a frequency of 800-1200 rpm for 2 minutes.

### EXAMPLE VII

In a coated collagen sponge containing human fibrinogen, bovine thrombin and aprotinin, the stability of the coating suspension was investigated for the duration of a coating process of 7 hours under environmental conditions of the production rooms, e.g. at 2-8° C.:

Each active ingredient was assayed at different sampling times. The results are shown in table I below.

TABLE I

| | Human fibrino- | Bovine thrombin | Aprotinin |
|---|---|---|---|
| | gen (mg/ml) | (I.U./ml) | (Ph. Eur. U/ml) |
| At start of coating | 50.0 | 21.8 | 0.59 |
| After 4.5 hours | 49.7 | 23.3 | 0.59 |
| After 7 hours (end | 49.1 | 20.4 | 0.56 |
| of coating process) | | | |

The results show satisfactory stability for all three components.

### EXAMPLE VIII

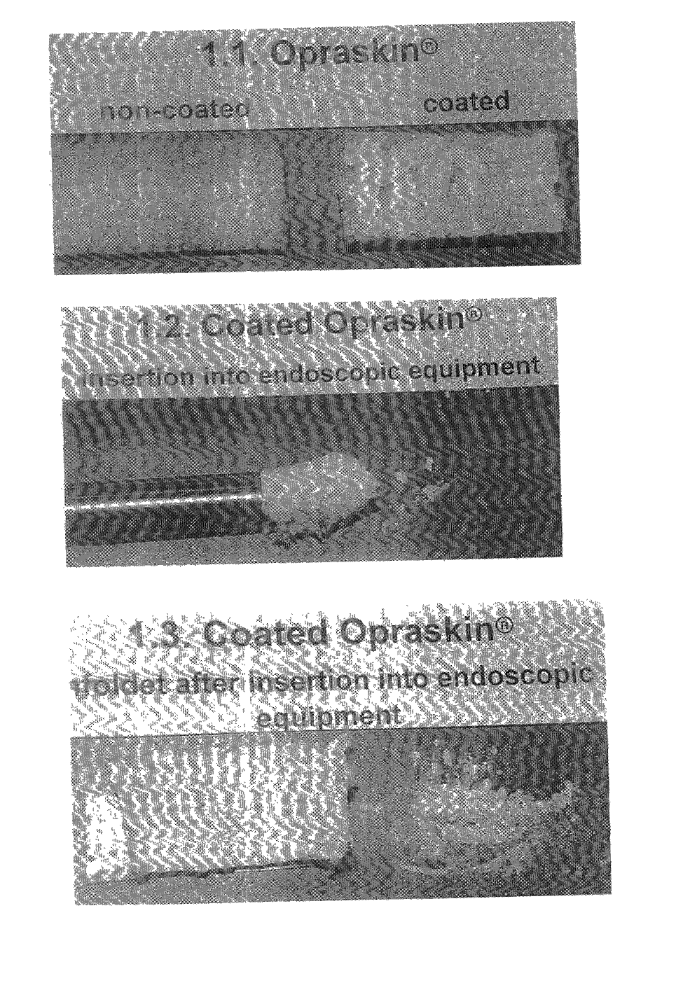

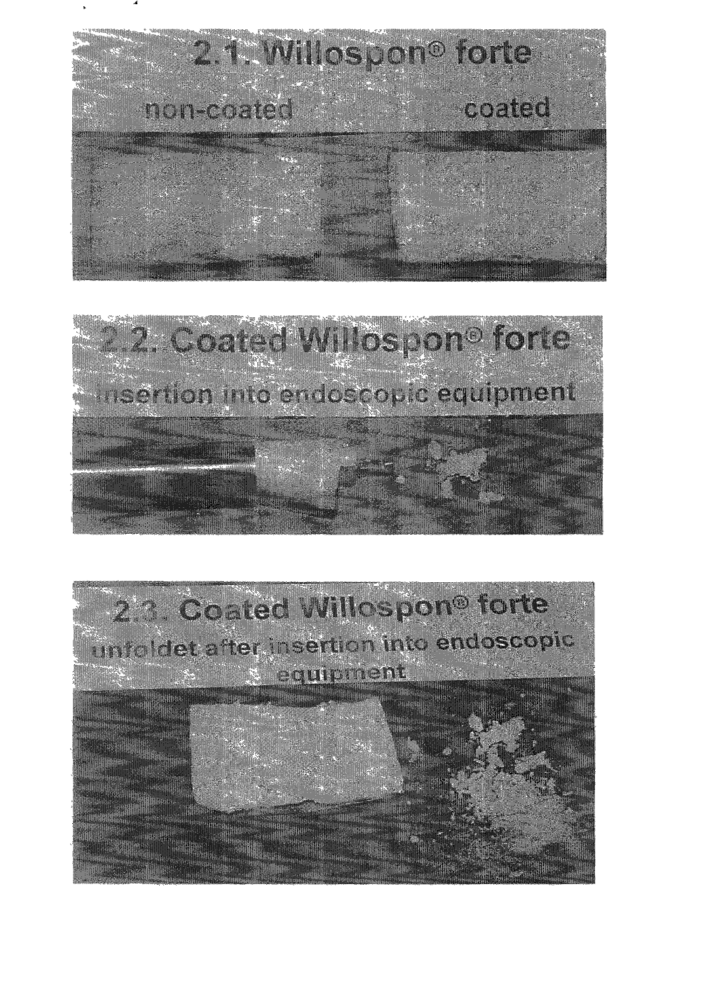

Reference is made to FIGS. 1-7, wherein the various figures show:

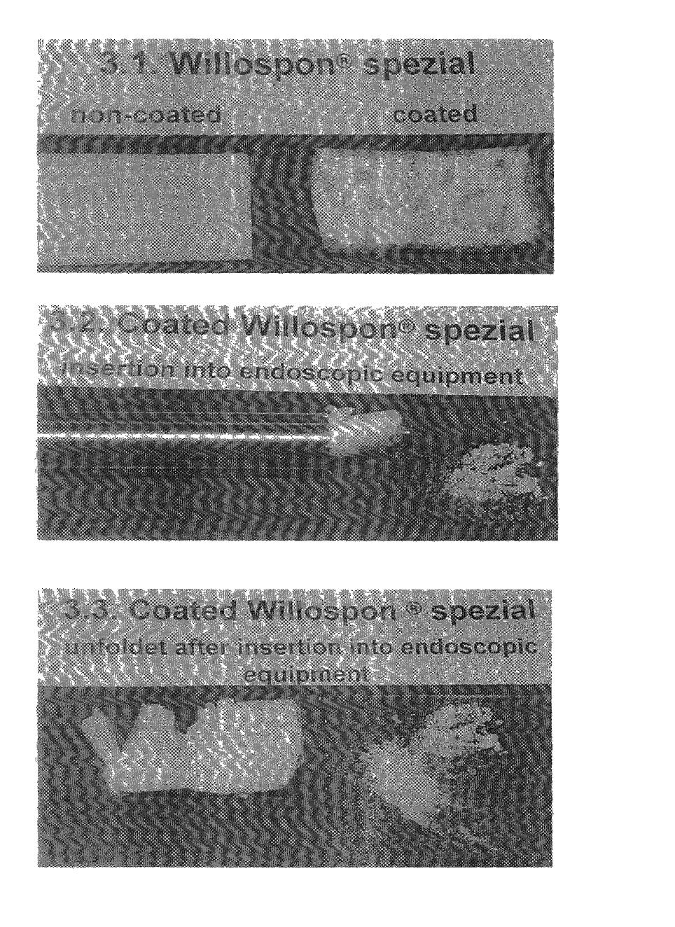

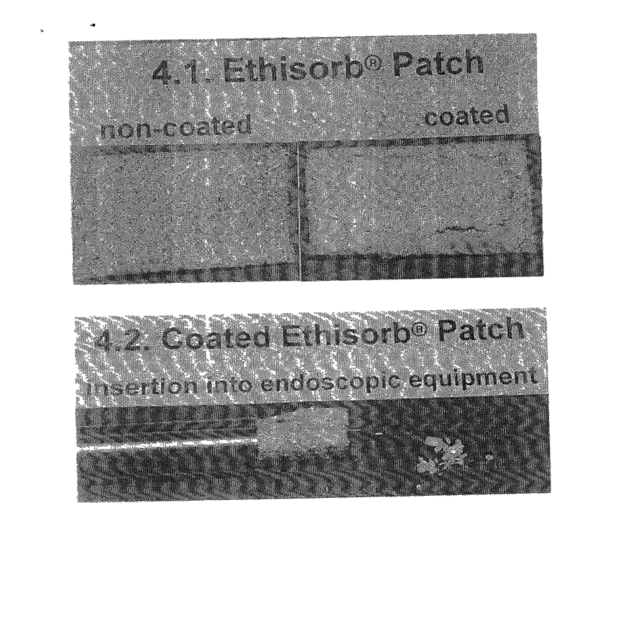

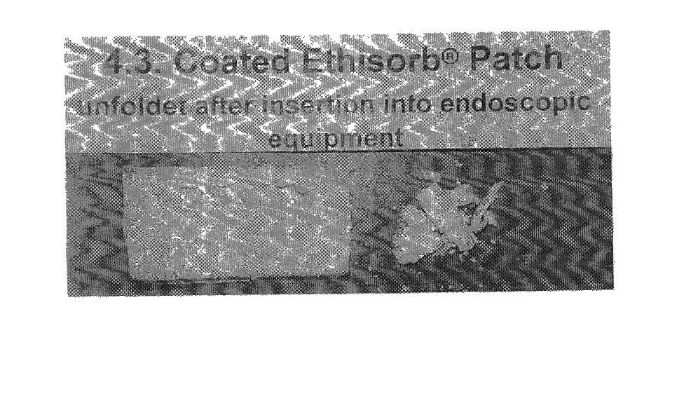

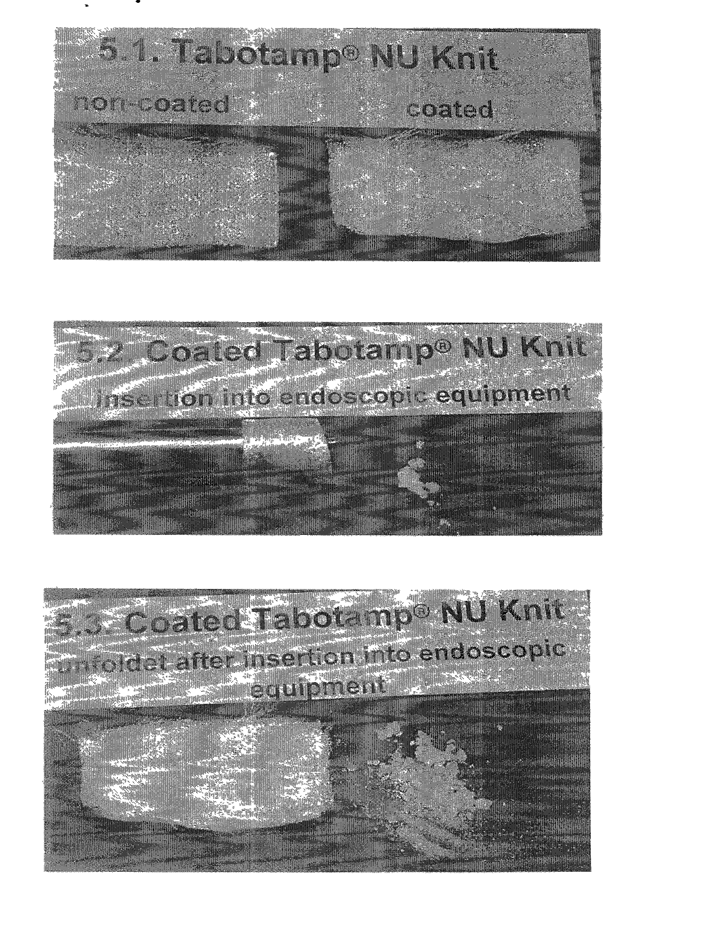

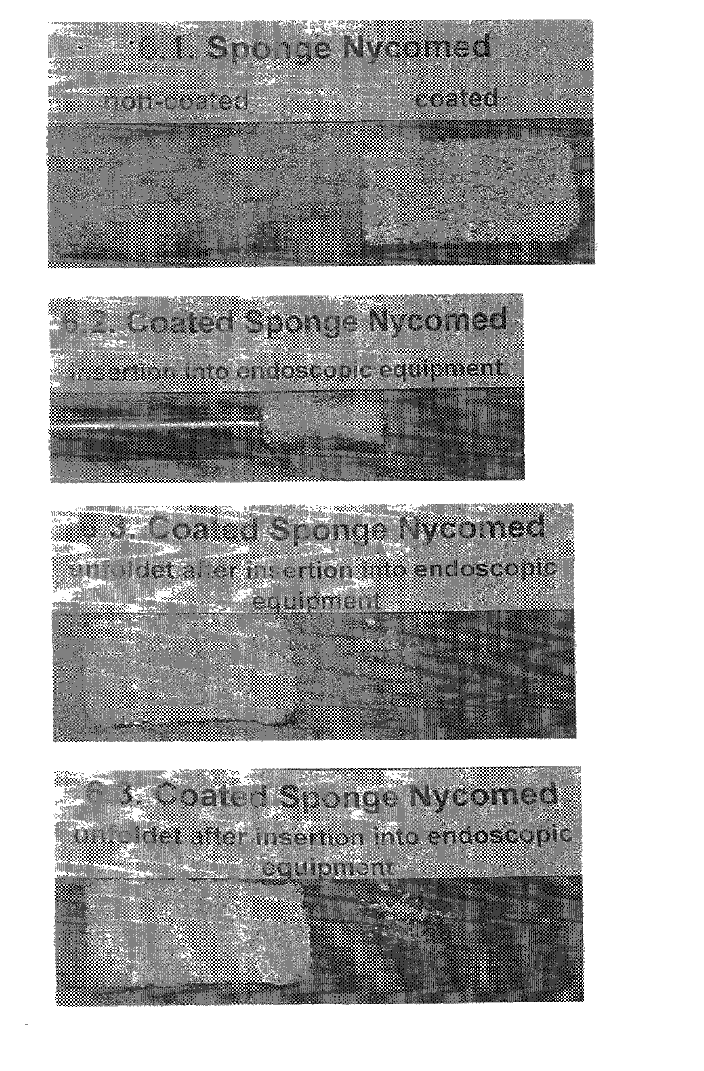

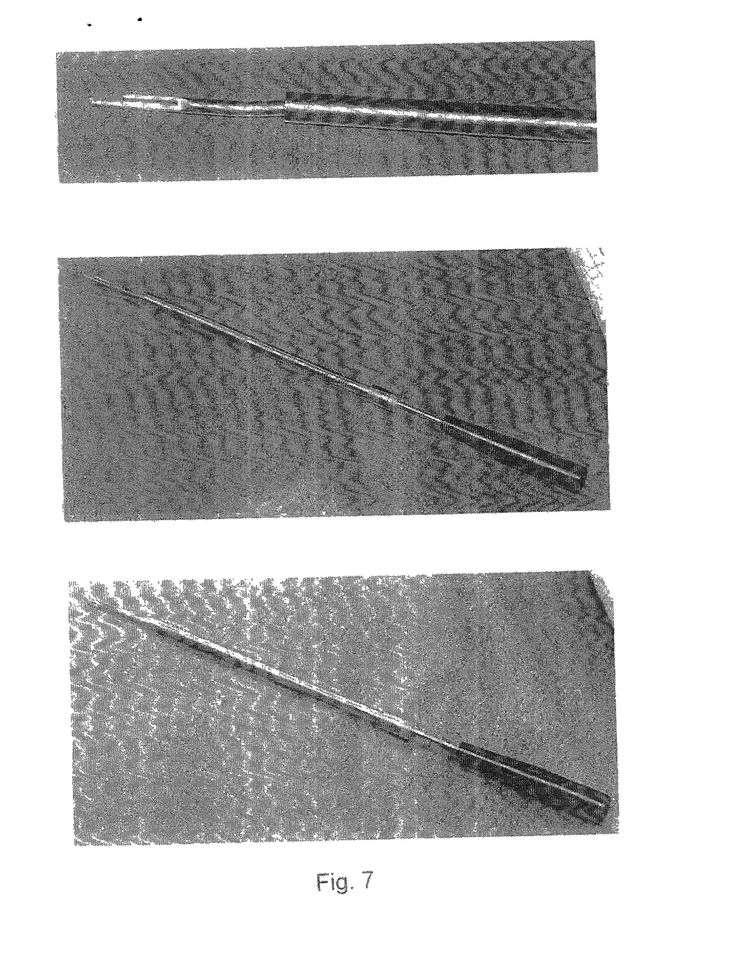

FIG. 1
1.1 Opraskin ®: non-coated/coated
1.2 Coated Opraskin ®: insertion into endoscopic equipment
1.3 Coated Opraskin: unfolded after insertion into endoscopic equip.
FIG. 2
2.1 Willospon ® forte: non-coated/coated
2.2 Coated Willospon ® forte: insertion into endoscopic equipment
2.3 Coated Willospon ® forte: unfolded after insertion into endoscopic
equipment
FIG. 3
3.1 Willospon ® Spezial: non-coated/coated
3.2 Coated Willospon ® Spezial: insertion into endoscopic equipment
3.3 Coated Willospon ® Spezial:: unfolded after insertion into endoscopic
equipment
FIG. 4
4.1 Ethisorb ® Patch: non-coated/coated
4.2 Coated Ethisorb ® Patch: insertion into endoscopic equipment
4.3 Coated Ethisorb ® Patch: unfolded after insertion into endoscopic
equipment
FIG. 5
5.1 Tabotamp ® NU Knit: non-coated/coated
5.2 Coated Tabotamp ® NU Knit: insertion into endoscopic equipment
5.3 Coated Tabotamp ® NU Knit: unfolded after insertion into endoscopic
equipment
FIG. 6
6.1 Sponge Nycomed: non-coated/coated [lab sample]
6.2 Coated sponge Nycomed [lab sample]: insertion into endoscopic
equipment
6.3 Coated collagen sponge Nycomed [lab sample]: unfolded after
insertion into endoscopic equipment
6.3 Coated collagen sponge Nycomed [production sample =
TachoComb ®]: unfolded after insertion into endoscopic equipment
FIG. 7
endoscopic tool: Endodock ®
endoscopic tool: Endodock ®
endoscopic tool: Endodock ®

Comparison of Coated Nycomed Sponge (TachoComb S) with Other Carrier Products Coated Identically as TachoComb S.

Adhesion of the Layer

Procedure

1. Coating of different carriers

| Brand name | Material | Manufactured/Distributed by |
|---|---|---|
| Opraskin ® | Collagen sponge | Lohmann, Postfach 2343, D- |
| | | 56513 Neuwied |
| Willospon ® | Collagen sponge | Will-Pharma, Postbus 30, |
| forte | (calves) | NL 1160 AA Zwanenburg |
| Willospon ® | Gelatine sponge | Will-Pharma, Postbus 30, |
| Special | | NL 1160 AA Zwanenburg |
| Ethisorb ® Patch | Polyglactin910/ | Johnson/Johnson |
| | Polydioxanon | (manufacturer) |
| | | Ethicon, Robert-Koch-Str. 1 |
| | | D-22851 Norderstedt |
| Tabotamp ® | Oxidized regenerated | Johnson/Johnson |
| NU Knit | cellulose | (manufacturer) |
| | | Ethicon, Robert-Koch-Str. 1 |
| | | D-22851 Norderstedt |
| Collagen sponge | Equine collagen | Nycomed Austria |
| Nycomed | sponge | Sankt-Peter-Str. 25 |
| | | A-4021 LINZ |

An area of 2×4,5 cm² of each carrier was coated with TachoComb S coating suspension. The amount of coating suspension corresponded to TachoComb specification (5,5 mg fibrinogen/cm²). The samples were dried.

2. A sample of 1×4, 5 cm² was prepared of each coated carrier.

3. The adhesion of the layer was tested as follows

Method Description

Apparatus

Analytical balance (measurement precision ±0.5 mg)

Vibrofix shaker combined with fixation device

Ruler with millimeter graduation

Stop-watch, scalpel, tubes of 2 cm internal diameter with stopper

Procedure

The procedure and calculation for determining abrasion are described above.

Results

| Carrier | Substance | Abrasion (mg/cm²) |
|---|---|---|
| Opraskin ® | lyoph. Collagen | 2, 1 |
| Willospon ® forte (3 mm) | lyoph. Collagen | 1, 2 |
| Willospon ® Spezial | Gelatine | 2, 1 |
| (1 mm) | | |
| Ethisorb ® Patch | Polyglactin/dioxanon | 14, 3 |
| (ZVP609) | | |
| Tabotamp ® NU Knit | oxidized cellulose | 9, 2 |
| Collagen sponge Nycomed | collagen, foamed | 0, 15 |

Comment

All carriers except Nycomed collagen sponge are not flexible after coating.

The sample has to be cut out very cautiously. If it is cut out by using a pair of scissors a lot of the coating material will flake off because the layer in itself is rigid. Ethisorb® patch showed almost no connection with the coating material at all. When shaken a little bit, all of the coating peels off like a “carpet”.

The difference between Nycomed collagen sponge and the other carrier materials shows quite clearly.

Elasticity of the Moistened Coated Carrier

Procedure

1. Coating of different carriers

An area of 2×4,5 cm² of each carrier was coated with TachoComb S coating suspension. The amount of coating suspension corresponded to TachoComb specification (5,5 mg fibrinogen/cm²). The samples were dried.

2. A sample of about 5-7 cm² was prepared of each coated carrier. The exact starting area of the dry sample was determined.

3. The sample was moistened and put on an elastic Latex sheet fixed to a special equipment as described in detail under the heading “procedure”. Then pressure was put on the Latex sheet which expanded. After 2 times of expansion and relaxation the sheet is expanded for a third time. The area of the carrier was measured at the highest expansion point.

Method Description

Apparatus/Chemicals

Peristaltic pump (IKA PA-SF)

Pressure buffering bottle (3 outlets)

VDO manometer (0-250 mbar)

Glass funnel (Ø opening1: 30 mm, opening 2: 15 mm)

Silicone tubings and clamps, Latex gloves (Semper med), scalpel, ruler with millimeter graduation, scissors

Physiological saline

Procedure

The following equipment is connected air tight to the three outlets of the pressure buffering bottle via silicone tubings:

a) peristaltic pumpe

b) manometer

c) glass funnel/opening 2

A double sheet of about 8×8 cm² is cut from a Latex glove. This sheet is fixed airtight to the glass funnel/ opening 1.

About 5-7 cm² coated area are cut out of the coated carrier using a scalpel.

The area of the sample is measured (starting area). The coating of the sample is moistened with saline and placed on the Latex sheet. Then it is pressed to the Latex sheet manually for about 1 min.

Using the peristaltic pump the Latex sheet is expanded by putting on a pressure of about 70 mbar. This is repeated twice with relaxation of the Latex sheet afterwards. At the third expansion the area (length and width) of the coated carrier is measured at the highest point of Latex sheet expansion.

Calculation:

$${}_{″} {Elasticity}^{″} = \frac{area of the carrier at third expansion}{starting area of sample}$$

Results

| Carrier | Substance | Elasticity factor |
|---|---|---|
| Opraskin ® | lyoph. collagen | 1, 78 |
| Willospon ® forte (3 mm) | lyoph. collagen | 1, 53 |
| Willospon ® Spezial (1 mm) | gelatine | 1, 79 |
| Ethisorb ® Patch (ZVP609) | Polyglactin/dioxanon | 1, 0 |
| Tabotamp ® NU Knit | oxidized cellulose | 1, 15 |
| Collagen sponge Nycomed | collagen, foamed | 1, 55 |

Comment:

The elasticity of the moistened Collagen sponge Nycomed (TachoComb) is one of the important characteristics of the product. Elasticity is essential in thoracic and abdominal surgery. After gluing the carrier should be able to follow for example expansion and relaxation movements of the lungs or intestines. Especially Ethisorb® showed no elasticity at all. It detached from the coating immediately. Coated Willospon® Spezial and Opraskin® showed structural defects during the test.

Use of Coated Carrier in Endoscopic Surgery

Procedure:

1. Coating of different carriers

An area of 2×4 cm² of each carrier was coated with TachoComb S coating suspension. The amount of coating suspension corresponded to TachoComb specification (5,5 mg fibrinogen/cm²). The samples were dried.

2. The handling of the coated carrier samples for use in endoscopic surgery and the loss of coating due to this handling are documented by a digital photo-equipment.

Method Description

Apparatus

Endodock: Endoscopic tool designed for the use of TachoComb® in endoscopic surgery (see FIG. 7). Digital photo-equipment.

| Carrier | Carrier material |
|---|---|
| Opraskin ® | lyoph. collagen |
| Willospon ® forte (3 mm) | lyoph. collagen |
| Willospon ® Spezial (1 mm) | gelatine |
| Ethisorb ® Patch (ZVP609) | Polyglactin/dioxanon |
| Tabotamp ® NU Knit | oxidized cellulose |
| Collagen sponge Nycomed | collagen, foamed |

List of Investigated Carriers

Procedure

Picture Series Taken of Each Carrier:

1. Picture: Documentation of the non-coated and coated carrier samples.

2. Picture: The coated samples are inserted into the endoscopic equipment (Endodock).

The sample has to be flattened manually to be able to wrap it around a guiding “pin”. Then the sample is inserted carefully into the steel tube of 10 mm in diameter. Documentation of the sample partially inserted into the Endodock tube.

3. Picture: The sample is pushed out carefully. Afterwards the sample has to be unfolded.

The coating that has split of the carrier due to this handling is gathered beside the carrier. The unfolded sample after insertion into the endoscopic equipment and the loss of the coating due to this handling are documented.

Comment

TachoComb (coated equine collagen sponge/Nycomed) in endoscopic surgery is the most demanding application of the product. TachoComb is inserted into an endoscopic equipment. The tube of this equipment is generally 10-13 mm in diameter. To be inserted into the tube TachoComb is flattened and then wrapped around a guiding “pin” and then inserted carefully into the tube. Therefore the connection of the coating to the carrier and within itself has to be strong but the product has to stay flexible enough in dry condition to be bent and rolled up. When brought to the site of the surgery TachoComb is carefully pulled out of the tube. Then it has to be unwrapped and placed to the wound surface. This often requires some adjustments. Therefore adhesion of the layer to the carrier should be strong enough to withstand this handling.

Results

The results are seen from the enclosed FIGS. 1-6. An estimate of the cast of coating material is as follows:

| | |
|---|---|
| Opraskin ® | 30-40% |
| Willospon ® forte | 60-70% |
| Willospon ® Spezial | 50% |
| Tacotamp ® NU knit | 60-70% |
| Ethisorb ® Patch | 95% |
| Collagen sponge Nycomed | <5% |

As Ethisorb® is a very rigid carrier the adhesion of the coating is very bad. Therefore coated Ethisorb® lost almost all of the coating in this investigation. Compared to coated collagen sponge of Nycomed all the other investigated carriers have a flat surface to be coated. Therefore the coating lies like a “flat carpet” on the carrier. This leads to a rather unflexible structure of the dry coated carriers. Bending or rolling up often breaks the coating in itself.

After insertion of the coated carriers into the tube of the endoscopic equipment and the unfolding of the sample afterwards all carriers except collagen sponge of Nycomed lost quite a lot of the coating so that large areas are left without coating material.

The structure and texture of Nycomed collagen sponge is the basis of the high flexibility of TachoComb in dry or moistened conditions. Nycomed collagen sponge is foamed and has polygonal chambers inside. On the surface these chambers are cut to caverns. These caverns enlarge the coating surface. During coating the coating suspension is distributed evenly onto the structured surface. During the drying the solution containing both fibrinogen and thrombin is fixed as solids into the caverns. Therefore TachoComb can be cut to desired sizes and can be inserted into endoscopic equipment with only a small loss of coating material or no loss at all.

The high flexibility of dry TachoComb is a big advantage compared to all other investigated coated carriers.

Manufacture of Collagen Sponge

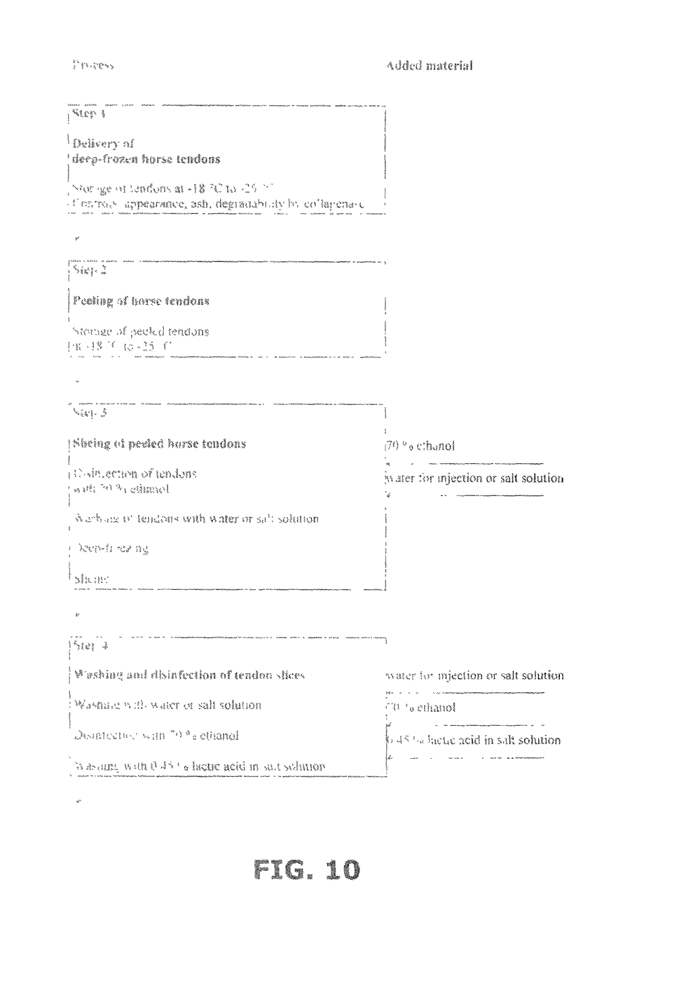

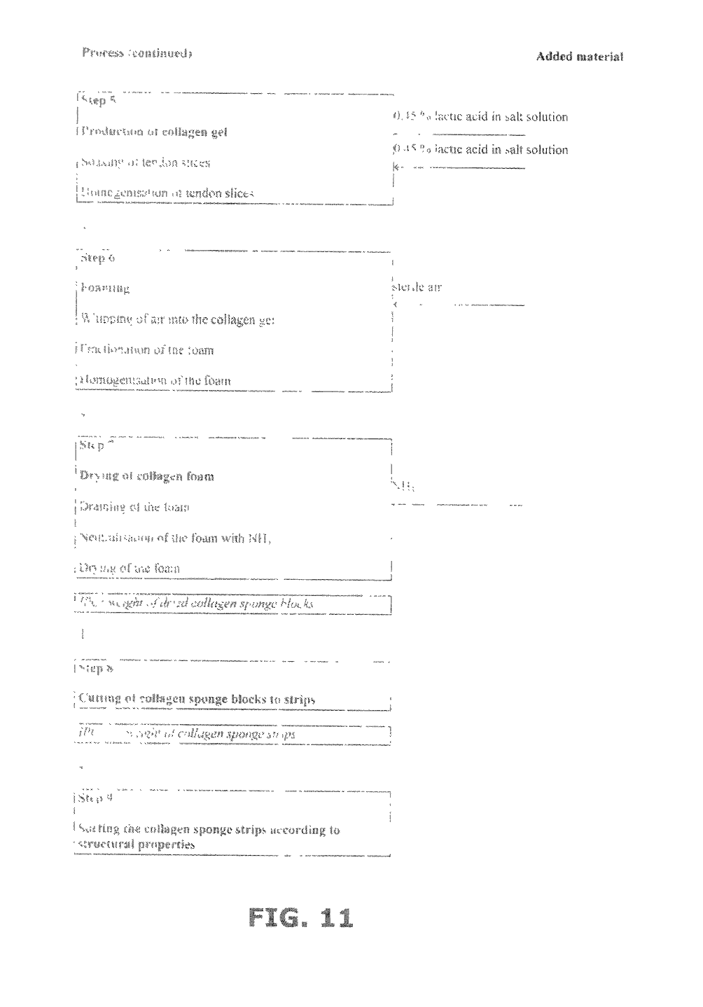

The collagen sponge referred to in the present text may be manufactured by a method as generally illustrated in FIGS. 10 and 11 and as described below:

It has been found that the successful coating of a collagen sponge with a fibrin glue preparation depends on the texture of the collagen sponge. It is thus desirable to provide a method of producing a collagen sponge with a certain texture, in particular with the aim of making the collagen sponge suitable for coating with a fibrin glue preparation, so as to obtain a material for healing and sealing wounds. It is further desirable to provide a method of producing a collagen sponge having improved physical characteristics in relation to prior art sponges, in the sense of improved humidity, elasticity, density and elasticity module. It is further desirable to provide a method for preparing a collagen sponge which is air and liquid tight in the sense that, once the collagen sponge is applied to a wound, it will not allow air or liquid to soak through the collagen sponge.

Thus, the method of preparing the collagen sponge, may comprise the steps of:

preparing a collagen gel,

mixing air into the collagen gel, so as to obtain a collagen foam,

drying the collagen foam, so as to obtain a dry block of collagen sponge having chambers therein,

isolating, from the block of collagen sponge, parts of sponge with a chamber diameter of more than 0.75 mm and less than 4 mm, or having a chamber diameter average of at most 3 mm.

In the present context, the term “chamber diameter” should be understood as the largest straight-line wall-to-wall distance in a chamber, i.e. as the largest diagonal straight-line distance of a chamber. The chambers may be of a polygonal shape, such as of an octagonal shape.

It has been found that a chamber diameter of more than 0.75 mm and less than 4 mm, or a chamber diameter average of at most 3 mm, renders the collagen sponge particularly useful for being coated with a fibrin glue preparation. Preferably, the collagen gel has a dry mass in the range of 2-20 mg dry mass per 1 g gel, such as 4-18 mg, such as 5-13 mg, such as 6-11 mg per 1 g gel. The dynamic viscosity of the collagen gel is preferably 2-20 Ncm, such as 4-10 Ncm, such as 6-8 Ncm. The collagen sponge preferably has a water content of not more than 20%, such as 10-15%, such as about 18%. The elasticity module of the collagen sponge is preferably in the range of 5-100 N/cm, such as 10-50 N/cm, and the density of the sponge is preferably 1-10 mg/cm³, such as 2-7 mg/cm

It has been found that a collagen sponge prepared by the above method is air and liquid tight in the sense that, once the collagen sponge is applied to a wound, it will not allow air or liquid to pass through the collagen sponge. Liquids are absorbed in the sponge. This effect is primarily achieved due to the fact that the step of mixing air into the collagen gel provides a collagen sponge which has a three-dimensional structure with stacked chambers separated and substantially totally enclosed by walls of collagen material, in contradiction to those known collagen sponges which have a fibre structure.

The collagen gel may comprise material of different types, such as type I, II or III from mammalian, transgenic or recombinant sources, but all other types of collagen can be used. The collagen may comprise material from tendons selected from the group consisting of equine tendons, human tendons, and bovine tendons. The collagen gel may additionally or alternatively comprise recombinant collagen material.

The collagen content of the isolated parts of sponge is preferably 50% -100% related to dry mass of the sponge, such as 75% -100%, such as 80% -100%, such as 85% -100%, such as 90% -1000/%, such as 92-100%, such as 92-98%, such as 93-97%, such as 94% -960%.

The step of preparing the collagen gel preferably comprises the steps of:

storing the tendons at a temperature between −10° C. and −30° C., and peeling the tendons,

removing foreign protein from the tendons,

reducing germ content in the tendons,

swelling the tendons,

homogenising the swelled tendons.

The steps of storing, peeling, removing protein, reducing of germ content, and swelling aim at purifying the raw material, whereas the step of homogenising aims at obtaining the collagen in the form of a gel.

The step of reducing of germ content preferably comprises adding an acid, such as an organic acid, such as lactic acid to the tendons. Further, an organic solvent, such as an alcohol, such as ethanol is preferably added to the tendons. Further, the step of swelling of the tendons preferably comprises adding lactic acid to the tendons. The lactic acid used may be a 0.40-0.50% lactic acid, such as a 0.45% lactic acid.

The step of swelling of the tendons may comprise storing the tendons at a temperature of 4° C. to 25° C., such as a temperature of 10° C. to 20° C., for a period of 48 to 200 hours, such as a period of 100 to 200 hours.

The step of homogenising the swelled tendons is preferably carried our so as to obtain a particle size of collagen gel fragments, i.e. fibre balls, with a diameter of 0.8-1.2 cm, such as approximately 1 cm. Further, the physical characteristics of the collagen gel are preferably as stated above. The appropriate characteristics may for example be achieved by performing the step of homogenising the swelled tendons by means of a toothed disk mill or adequate homogenisation equipment.

The step of mixing air into the collagen gel preferably comprises the steps of:

mixing ambient air into the gel by means of a mixer so as to generate a collagen foam,

feeding the mixed gel foam into a fractionising channel,

separating collagen gel and collagen foam contained in the fractionising channel.

At least some of the collagen gel separated from the collagen foam in the fractionising channel may be led back to the mixer. In that case, the ratio between the amount of collagen gel which is led back to the mixer from the fractionising channel and the amount of fresh collagen gel led to the mixer is preferably between 0.1 and 0.5. The step of separating collagen gel and collagen foam preferably comprises the steps of:

separating a selected part of the collagen foam contained in the fractionising channel,

leading the selected part of the collagen foam out of the fractionising channel for drying thereof.

In a preferred embodiment of the method, a temperature of 15° C. to 40° C., such as 20° C. to 25° C. is maintained in the fractionising channel.

Subsequent to mixing air into the collagen gel, the collagen foam may be homogenised for a period of 2 to 4 minutes.

Prior to the step of drying the collagen foam and subsequent to the step of mixing air into the collagen gel, a neutraliser may be added to the collagen foam, and the collagen foam is preferably neutralised in order to arrive from a pH-value of, usually, between 2.5 and 3.5 to a pH-value in the collagen foam between 6.5 and 8.5. A neutraliser comprising an ammonia solution may be used, and the collagen foam is preferably neutralised for a period of 5-30 hours, such as 10-20 hours, such as approximately 24 hours.

Prior to the step of drying the collagen foam, the collagen foam is preferably filled into a drying container in such a way that substantially no air is drawn into the foam while filling.

The step of drying preferably comprises drying at a temperature between 15° C. and 60° C., such as between 20 and 40° C., for a period of 50-200 hours, such as 100-150 hours, so as to obtain a dry collagen sponge. The drying may be performed at a pressure slightly under atmospheric pressure, such as at a pressure of between 700 and 900 mbar, such as approximately 800 mbar.

The collagen sponge produced by the above method preferably fulfils at least one of the following criteria:

pH-value between 5.0 and 6.0,

lactic acid content at the most 5%,

ammonium content at the most 0.50%,

soluble protein content, calculated as albumin content, at the most 0.5%,

sulphate ashes content at the most 1.00%,

heavy metal content at the most 20 ppm,

microbiological purity, at the most 10³ CFU/g,

collagen content of 75% to 100%,

density of 1-10 mg/cm³, such as 2-7 mg/cm³,

elasticity module of 5-100 N/cm, such as 10-50 N/cm.

The step of isolating parts of collagen sponge may comprise dividing the collagen sponge into a plurality of parts by cutting. The parts obtained may be shaped in any desirable form, such as conical, cylindrical, including cylindrical with an annular cross-section, rectangular, polygonal, cubic, and flat sheets or they may be transformed into a granulate by an appropriate granulating method etc.

As it is apparent from the above, the collagen sponge may be produced by a method, comprising the steps of:

preparing a collagen gel,

mixing air into the collagen gel, so as to obtain a collagen foam,

drying the collagen foam, so as to obtain a dry block of collagen sponge having chambers therein,

isolating, from the block of collagen sponge, parts of sponge having the following properties:

elasticity module in the range of 5 to 100 N/cm,

density in the range of 1 to 10 mg/cm³,

chamber diameter of more than 0.75 mm and less than 4 mm, or a chamber diameter average of at most 3 mm.

1. A suspension comprising fibrinogen, thrombin and alcohol, the suspension having been obtained by a method comprising:
providing a fibrinogen mixture of fibrinogen and an alcohol,
providing a thrombin mixture of thrombin and an alcohol,
mixing the fibrinogen mixture and the thrombin mixture, so as to obtain said suspension,
the suspension containing fibrinogen and thrombin particles, the Folk Ward mean diameter of the particles being 25-100 μm.

2. A suspension according to claim 1, wherein the Folk Ward mean diameter of the particles is 35-80 μm.

3. A suspension according to claim 1, wherein the viscosity of the suspension is so that a volume of 90-120 ml of suspension, when influenced by gravity only, exits through a bottom opening of a container having
a cylindrical portion with an inner diameter of 40-50 mm and a height of 55-65 mm, and
a conical bottom portion with a height of 17-23 mm, whereby the bottom opening is provided at the lower end of the conical portion as a circular opening with a diameter of 2-3 mm,
in 25-75 seconds.

4. A suspension according to claim 3, wherein said volume of suspension exits through the bottom opening in 30-50 seconds.

5. A suspension according to claim 1, further comprising aprotinin.

6. A method of preparing a suspension with fibrinogen and thrombin, comprising:
providing a fibrinogen mixture of fibrinogen and an alcohol,
providing a thrombin mixture of thrombin and an alcohol,
mixing the fibrinogen mixture and the thrombin mixture, so as to obtain said suspension,
so as to obtain a suspension containing fibrinogen and thrombin particles, the Folk Ward mean diameter of the particles being 25-100 μm.

7. A method according to claim 6, wherein, at the step of providing the fibrinogen mixture, the fibrinogen is pre-micronised so as to obtain particles having a Folk Ward mean diameter of 25-100 μm.

8. A method according to claim 6, wherein the pre-micronised fibrinogen is stirred into the alcohol to obtain said fibrinogen mixture.

9. A method according to claim 6, wherein, at the step of providing the mixture, the mixture is homogenized.

10. A method according to claim 9, wherein the mixture is homogenized at a temperature between 0° C. and 12° C.

11. A method according to claim 10, wherein the mixture is homogenized at a temperature between 2° C. and 8° C.

12. A method according to claim 11, wherein the temperature is lowered during homogenization.

13. A method according to claim 6, wherein the thrombin comprises at least one of: human thrombin, bovine thrombin, and recombinant thrombin.

14. A method according to claim 6, wherein the fibrinogen comprises at least one of: human fibrinogen and recombinant fibrinogen.

15. A method according to claim 6, wherein the suspension further comprises aprotinin.

16. A method according to claim 6, wherein the alcohol is an ethanol.

17. A method according to claim 16, wherein the ethanol is an anhydrous ethanol.

18. A method according to claim 6, wherein the step of mixing fibrinogen mixture and the thrombin mixture is carried out while stirring the suspension.

19. A method according to claim 18, wherein the stirring is carried out at a temperature between 0° C. and 12° C.

20. A method according to claim 19, wherein the stirring is carried out at a temperature between 20 and 8° C.

21. A method for coating a carrier with a suspension comprising fibrinogen and thrombin, wherein the suspension has been derived from a method comprising the steps of:
providing a fibrinogen mixture of fibrinogen and an alcohol,
providing a thrombin mixture of thrombin and an alcohol,
mixing the fibrinogen mixture and the thrombin mixture, so as to obtain said suspension,
so as to obtain a suspension containing fibrinogen and thrombin particles, the Folk Ward means diameter of the particles being 25-100 μm, the method of coating comprising:
providing the suspension of fibrinogen, thrombin and an alcohol at a location near the carrier,
applying said suspension to a coating surface of the carrier.

22. A method according to claim 21, wherein the carrier is a collagen carrier.

23. A method according to claim 22, wherein the collagen carrier is a collagen sponge.

24. A method according to claim 23, wherein the collagen sponge fulfils at least one of the following criteria:
pH-value between 5.0 and 6.0,
lactic acid content at the most 5%,
ammonium content at the most 0.5%,
soluble protein content, calculated as albumin content, at the most 0.5%,
sulphate ashes content at the most 1.0%,
heavy metal content at the most 20 ppm,
microbiological purity, at the most 10³ CFU/g,
collagen content of 75 to 100%,
density of 1 to 10 mg/cm³,
elasticity module in the range of 5-100 N/cm.

25. A method according to claim 23, wherein the carrier is a collagen sponge, and wherein the collagen sponge has been derived from a method comprising the steps of: preparing a collagen gel,
mixing air into the collagen gel, so as to obtain a collagen foam,
drying the collagen foam, so as to obtain a dry block of carrier having chambers therein,
isolating, from the block of collagen sponge, parts of sponge showing at least one of:
a chamber diameter of more than 0.75 mm and less than 4 mm, and
chambers of an average diagonal dimension of 3 mm.

26. A method according to claim 21, wherein the step of applying the suspension to the carrier is performed at an ambient temperature of 0°-12° C.

27. A method according to claim 26, wherein the step of applying the suspension to the carrier is carried out at an ambient temperature of 1°-10° C.

28. A method according to claim 27, wherein the step of applying the suspension to the carrier is carried out at an ambient temperature of 2°-8° C.

29. A method according to claim 21, wherein the step of applying the suspension to the carrier is carried out in an ambient atmosphere with a relative humidity of 75-99%.

30. A method according to claim 29, wherein the step of applying the suspension to the carrier is carried out in an ambient atmosphere with a relative humidity of 85-95%.

31. A method according to claim 21, wherein a volume of 0.08 ml-0.12 ml of suspension is applied to the carrier pr. cm² of the coating surface.

32. A method according to claim 21, wherein the suspension is distributed evenly over a given width of the coating surface, so that the mass of fibrinogen per area unit of the coating surface varies at most 250%.

33. A method according to claim 21, wherein an applicator comprising at least one jet is used for applying the suspension to the carrier, whereby the suspension is forced through the jet while the carrier and the jet are moved relative to each other.

34. A method according to claim 21, wherein an applicator comprising a container having a plurality of separate outlets is used for applying the suspension to the carrier, and wherein the suspension is forced from the container through the outlets onto the carrier.

35. A method according to claim 34, wherein the carrier and the applicator are moved relative to each other in a transport direction while the suspension is being applied to the carrier.

36. A method according to claim 35, wherein the rate of movement is 0.025 ml-0.05

37. A method according to claim 36, wherein the rate of movement is 0.03-0.04 ml.

38. A method according to claim 34, wherein the flow rate of suspension applied to the carrier through the applicator is 400-600 ml/min.

39. A method according to claim 38, wherein the flow rate is 470-550 ml/min.

40. A method according to claim 39, wherein the flow rate is 495-505 ml/min.

41. A method of drying a suspension of fibrinogen, thrombin and an alcohol applied as a wet coating on a coating surface of a carrier, the method comprising the step of:
submitting the coated carrier to a pressure below 1000 mbar, so as to obtain a dried coating surface on the carrier, so as to fixate the dried coating to the coating surface.

42. A method according to claim 41, wherein the suspension has been obtained by:
providing a fibrinogen mixture of fibrinogen and an alcohol,
providing a thrombin mixture of thrombin and an alcohol,
mixing the fibrinogen mixture and the thrombin mixture, so as to obtain said suspension,
and wherein the carrier is a collagen sponge which has been derived from a method comprising the steps of:
preparing a collagen gel,
mixing air into the collagen gel, so as to obtain a collagen foam,
drying the collagen foam, so as to obtain a dry block of collagen sponge having chambers therein,
isolating, from the block of collagen sponge, parts of sponge showing at least one of:
a chamber diameter of more than 0.75 mm and less than 4 mm, and
a chamber with an average diagonal dimension of 3 mm,
and wherein the coating has been applied to the collagen sponge by:
providing the suspension of fibrinogen, thrombin and an alcohol at a location near the collagen sponge,
applying the suspension to the coating surface of the collagen sponge.

43. A method according to claim 41, wherein the coated carrier is submitted to said pressure at a temperature of 0° C.-12° C.

44. A method according to claim 43, wherein the coated carrier is submitted to said pressure at a temperature of 1° C.-10° C.

45. A method according to claim 44, wherein the coated carrier is submitted to said pressure at a temperature of 2° C.-8° C.

46. A method according to claim 41, wherein the coated carrier is submitted to said pressure at a relative humidity of the surrounding atmosphere of 75-99%.

47. A method according to claim 41, wherein the coated carrier is submitted to said pressure at a relative humidity of the surrounding atmosphere of 85-95%.

48. A method according to claim 41, wherein flow of air passes across the coated carrier during drying.

49. A method according to claim 43, wherein the coated carrier is kept at said temperature for a period of at least 1 hour.

50. A method according to claim 43, wherein the coated carrier is kept at said temperature for a period of at least 2 hours.

51. A method according to claim 43, wherein the coated carrier is kept at said temperature for a period of at least 4 hours.

52. A method according to claim 41, wherein the area of the dried coating surface is at least 75% the size of the area of the wet coating surface.

53. A method according to claim 41, wherein the area of the dried coating surface is at least 80% the size of the area of the wet coating surface.

54. A method according to claim 41, wherein the carrier and the dried coating surface have a water content not exceeding 12% by weight.

55. A method according to claim 41, wherein the carrier and the dried coating surface have a water content not exceeding 8% by weight.

56. A method according to claim 41, wherein the suspension further comprises aprotinin.

57. A coated collagen sponge with a coating of fibrinogen and thrombin, wherein the coated collagen sponge has been obtained by a method comprising the steps of:
providing a collagen sponge by a method comprising:
preparing a collagen gel,
mixing air into the collagen gel, so as to obtain a collagen foam,
drying the collagen foam, so as to obtain a dry block of collagen sponge having chambers therein,
isolating, from the block of collagen sponge, parts of sponge showing at least one of:
a chamber diameter of more than 0.75 mm and less than 4 mm, and
a chamber with an average diagonal dimension of 3 mm,
applying a suspension of fibrinogen, thrombin and an alcohol to a coating surface of the collagen sponge, and
submitting the coated carrier to a pressure below 1000 mbar, so as to obtain a dried coating surface on the carrier, so as to fixate the dried coating to the coating surface,
the coated collagen sponge having at least one of the following properties:
the suspension is distributed evenly over a given width of the coating surface, so that the mass of fibrinogen per area unit of the coating surface varies at most 25%,
the abrasion of the coating is less than 2.0 mg/cm² when a sample of 1×5 cm² of the coated material is shaken in a test-tube on a Vibrofix shaker at a frequency of 800-1200 rpm for 2 minutes.

58. A coated collagen sponge according to claim 57, wherein the suspension has a water content of 20-80 mg/ml.

59. A coated collagen sponge according to claim 57, wherein the suspension has a water content of 24-32 mg/ml.

60. A coated collagen sponge according to claim 57, wherein the thrombin content of the suspension is 20-40 I.U./ml.

61. A coated collagen sponge according to claim 57, wherein said thrombin content is 24-33 I.U./ml.

62. A coated collagen sponge according to claim 57, wherein the thrombin content is 2-4 I.U./cm² in average over the coating surface.

63. A coated collagen sponge according to claim 57, wherein the thrombin content is 2.3-3.3 I.U./cm² in average over the coating surface.

64. A coated collagen sponge according to claim 57, wherein the thrombin content does not exceed 5 I.U./cm² at any location on the coating surface.

65. A coated collagen sponge according to claim 57, wherein the thrombin content does not exceed 3.8 I.U./cm² at any location on the coating surface.

66. A coated collagen sponge according to claim 57, wherein the microbiological purity of the coated carrier is 4 CFU/cm², at the most.

67. A coated collagen sponge according to claim 57, wherein the microbiological purity of the coated carrier is 2.25 CFU/cm², at the most.
